(19) United States
(12) Patent Application Publication
(10) Pub. No.: US 2018/0177735 A1
(43) Pub. Date: Jun. 28, 2018

(54) Stable Pharmaceutical Composition Of Phytonadione And A Novel Process For Preparation Thereof

(71) Applicant: Amneal Pharmaceuticals Company GmbH, Steinhausen (CH)

(72) Inventors: Jatin Gajjar, Ahmedabad (IN); Atul Kathiriya, Ahmedabad (IN); Dhaval Shah, Ahmedabad (IN)

(21) Appl. No.: 15/835,801

(22) Filed: Dec. 8, 2017

(30) Foreign Application Priority Data
Dec. 22, 2016 (IN) 201621043889

(51) Int. Cl. A61K 9/20; A61K 31/122; A61K 9/10

(52) U.S. Cl. CPC A61K 9/2068; A61K 31/122; A61K 9/10; A61K 9/2009; A61K 9/2018; A61K 9/2059; A61K 9/2013

(57) ABSTRACT

The present invention is related to a process of preparing a stable pharmaceutical composition of phytonadione comprising the steps of a preparing dispersion of phytonadione in a pharmaceutically acceptable binder; spraying/mixing the dispersion on pharmaceutically acceptable excipients and finally, formulating the composition into a pharmaceutically administrable dosage form. The present invention further relates to preparation of stable pharmaceutical composition of phytonadione which exhibits an improved bioavailability in comparison to marketed pharmaceutical composition of phytonadione. Moreover, the present invention relates to phytonadione pharmaceutical composition which remains stable as per ICH guidelines.

### FIELD OF THE INVENTION

The present invention relates to a process for preparing a stable pharmaceutical composition of phytonadione. The process comprises preparing a dispersion of phytonadione in a pharmaceutically acceptable binder. Furthermore, phytonadione pharmaceutical compositions remain stable as per ICH (International Council for Harmonisation of Technical Requirements for Pharmaceuticals for Human Use) guidelines.

### BACKGROUND OF THE INVENTION

Phytonadione (Vitamin K1, also known as phylloquinone, phytomenadione) is a clear, yellow to amber, viscous liquid. Phytonadione is insoluble in water, soluble in chloroform and slightly soluble in ethanol. Phytonadione is known to be used to treat Vitamin K deficiency. Phytonadione is marketed under the brand name Mephyton®. MEPHYTON is a 5 mg tablet of phytonadione indicated for various coagulation disorders caused by vitamin K deficiency or interference with vitamin K activity. MEPHYTON is available in tablet dosage form and has a limited oral bioavailability due to phytonadione's water insolubility which affects miscibility in the GI fluid.

U.S. Pat. No. 4,892,889 disclose a process for making a spray-dried vitamin powder comprising of fat-soluble vitamins. The four most common fat-soluble vitamins disclosed are vitamin A (retinol), vitamin D (calciferol), vitamin E (tocopherol), and vitamin K (phylloquinone and menaquinone). It further discloses the process which is carried out by combining a fat-soluble vitamin, a gelatin having a bloom number between 30 and 300, a water-soluble carbohydrate, and an effective amount of water to permit spray-drying.

U.S. Pat. No. 7,279,180 disclose a stable emulsions and dry powders of mixtures of fat-soluble vitamins. It further discloses a process for preparing stable mixtures of fat-soluble vitamins in a finely dispersed form. The suitable vitamins disclosed are vitamins A, D, E and/or K.

The aforementioned prior art teaches spray-dried powder mixtures of fat-soluble vitamins including phytonadione. Problems associated with preparation of phytonadione include handling, distribution, and processing due to the viscous nature of the API (active pharmaceutical ingredient).

There are additional difficulties in preparing phytonadione formulations due to the viscous nature and insolubility of phytonadione. It is difficult to achieve a free flowing powder by mixing the liquid active ingredient with solid pharmaceutical excipients. Further, direct mixing of phytonadione with solid pharmaceutical excipients is not feasible as it does not result in a homogeneous distribution of phytonadione in the formulation.

In addition, the compaction of liquid form of phytonadione with suitable excipients is challenging because viscous liquid tends to squeeze out on to the surface of formulation due to compression force. Further, increasing the compression force to achieve desired strength of formulation may compromise other properties of formulation like disintegration time and dissolution profile of the formulation.

Moreover, lipophilic molecules, such as vitamins, are poorly solubilized in the aqueous medium along the gastrointestinal tract, thereby leading to poor bioavailability. These vitamins include vitamin A, D, E and K that are not readily absorbed by body due to this poor solubility in an aqueous medium, i.e., the gastrointestinal fluid. The prior art formulations and processes do not overcome the problems associated with the preparation of pharmaceutical dosage form of phytonadione. Accordingly, there is need for a novel process for preparing a stable pharmaceutical composition of water-insoluble viscous phytonadione liquid with pharmaceutical excipients without having a problem during compaction process which would improve the bioavailability of phytonadione.

The inventors of the present invention have addressed the above mentioned problems by formulating a pharmaceutical composition with a novel process containing a dispersion of phytonadione in pharmaceutically acceptable binder, which maintains a pharmaceutical composition in stable form over a longer period of time and also leads to an improved bioavailability of the pharmaceutical composition in comparison to currently marketed MEPHYTON formulation.

### SUMMARY OF THE INVENTION

A stable pharmaceutical composition comprises phytonadione and a process prepares a stable pharmaceutical composition of phytonadione.

The present invention is related to a process of preparing a stable pharmaceutical composition of phytonadione, wherein the pharmaceutical composition remains stable as per ICH guidelines and exhibits an improved bioavailability in comparison to marketed pharmaceutical composition of phytonadione. Improved bioavailability is achieved in comparison to a comparative pharmaceutical composition that was made without using a dispersion of phytonadione in a pharmaceutically acceptable binder.

Specifically, the present invention is related to a process of preparing a stable pharmaceutical composition of phytonadione comprising: (a) preparing a dispersion of phytonadione in a pharmaceutically acceptable binder; (b) spraying or mixing the dispersion with one or more pharmaceutically acceptable excipients to form the stable pharmaceutical composition; and finally, (c) formulating the stable pharmaceutical composition into a pharmaceutically administrable dosage form. The stable pharmaceutical composition exhibits an improved bioavailability in comparison to marketed pharmaceutical composition of phytonadione.

Without intending to be bound by any particular theory of operation, it is believed that the presence of a dispersion of phytonadione in a pharmaceutically acceptable binder along with the spraying or mixing of one or more pharmaceutically acceptable excipients results in an improved bioavailability in comparison to the marketed pharmaceutical composition of phytonadione.

### BRIEF DESCRIPTION OF THE DRAWING

FIG. 1 shows the graph of Mean concentration vs Time (hr) for cis-phytonadione comparing Test formulations (T1 and T2) with Reference formulation (R)

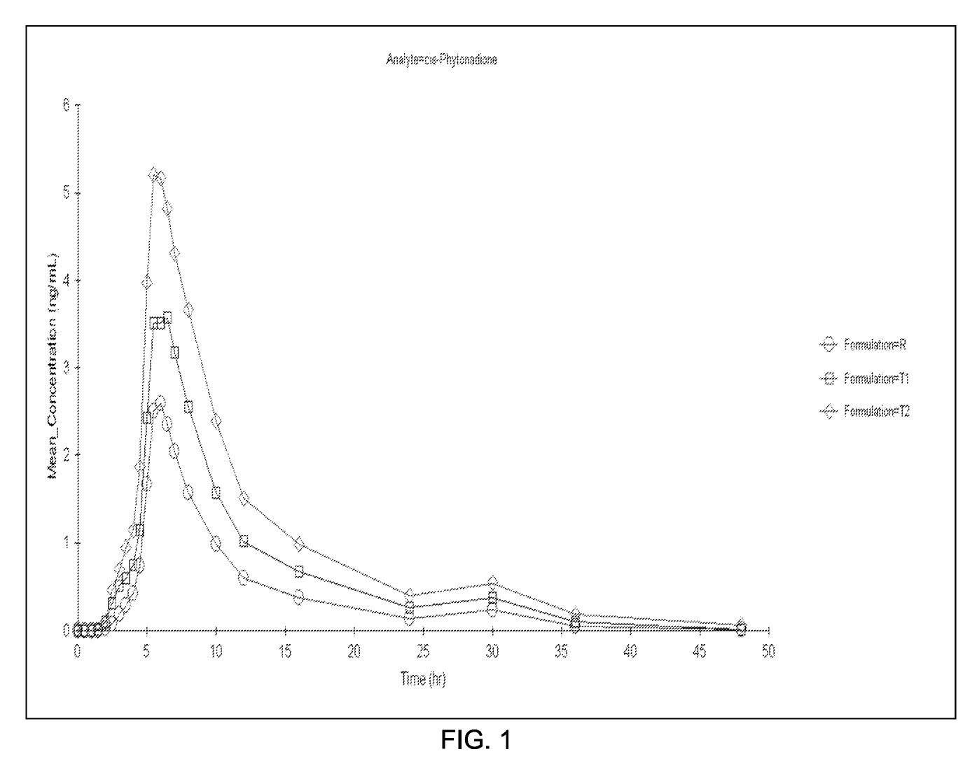

FIG. 2 shows the graph of Mean concentration vs Time (hr) for trans-phytonadione comparing Test formulations (T1 and T2) with Reference formulation (R)

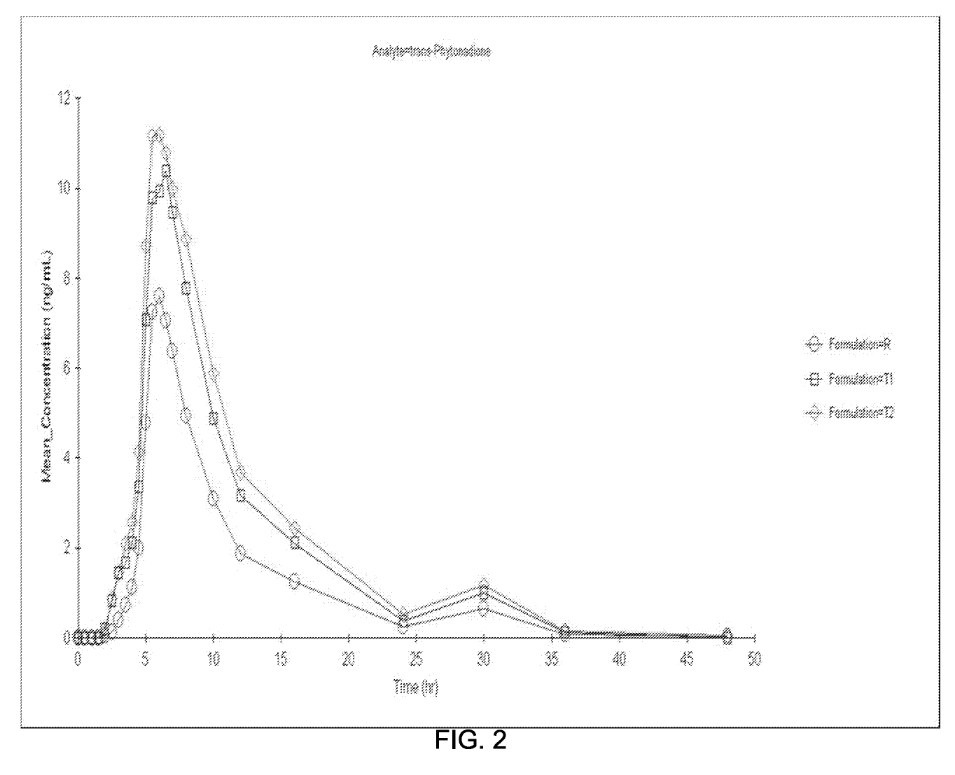

### DETAILED DESCRIPTION

The present invention relates to stable pharmaceutical compositions comprising phytonadione and a process for the preparation thereof.

The present invention specifically relates to a process of preparing phytonadione pharmaceutical compositions wherein the pharmaceutical compositions remain stable as per ICH guidelines.

Further, the present invention pharmaceutical composition provides improved bioavailability of phytonadione in comparison to currently marketed formulation.

The term “stable pharmaceutical composition” means a composition for pharmaceutical use which remains stable as per ICH guidelines.

The term “ICH guidelines” means composition remains stable for longer period of time at 25° C./60%+5% RH, 30° C./65%+5% RH, and 40° C./75%+5% RH conditions for a time period of at least 6 months.

The term “slightly soluble” means 1 g of material requires 100 to 1000 ml of solute to dissolve.

A first aspect of the present invention relates to a process of preparing a stable pharmaceutical composition of phytonadione comprising the steps of preparing dispersion of phytonadione in a pharmaceutically acceptable binder; spraying/mixing the dispersion on pharmaceutically acceptable excipients and finally, formulating the composition into a pharmaceutically administrable dosage form.

A second aspect of the present invention addresses the difficulties associated with the preparation of tablet dosage form of viscous liquid form of API. The preparation of physical mixture of liquid form of active pharmaceutical ingredient with solid pharmaceutical excipients to get free flow powder is difficult and cost ineffective. The direct mixing of phytonadione with solid pharmaceutical excipient is not feasible as it does not give homogeneous distribution of phytonadione in tablets dosage form.

A third aspect of the present invention is directed to dispersion of phytonadione with pharmaceutically acceptable excipients forms in powder form which makes compaction process simple for tablet dosage preparation purpose.

According to another embodiment of the present invention is directed towards a stable pharmaceutical composition comprising phytonadione and a binder, wherein the binder is present in an amount by weight relative to phytonadione from about 1:3 to 1:0.01, preferably from about 1:2 to 1:0.1, and more preferably from about 1:0.5. Preferable binder in present invention is acacia and may present in a weight ratio that is in the range of from about 1:2 to about 1:0.1.

According to another embodiment of the present invention, phytonadione is present in an amount from about 1% to about 10% w/w of composition, preferably from about 3% to about 8% w/w of composition.

According to another embodiment of the present invention provides a stable pharmaceutical composition containing phytonadione and one or more pharmaceutically acceptable excipients, wherein one or more pharmaceutically acceptable excipients selected from diluents, binders, disintegrants, glidants, lubricants, and/or mixtures thereof.

The binders used in the pharmaceutical composition of the present invention are selected from natural binders, synthetic binders, semisynthetic binders or a combination thereof. Specifically, the binders are selected from the group consisting of a polyvinylpyrrolidone, starch, cellulose derivatives like hydroxypropylmethyl cellulose, hydroxyethyl cellulose, hydroxypropyl cellulose, polyethylene glycol, acacia, gelatine, tragacanth, alginic acid, and the like. According to the present invention, preferable binders are acacia and/or starch and may present in an amount from about 1% to 20% by weight of composition, preferably from about 2% to 15% by weight of composition, more preferably from about 5% to 10% by weight of composition.

The diluents used in the pharmaceutical composition of the present invention are selected from the group consisting of: an inorganic phosphates like dibasic calcium phosphate, or sugars or sugar analogues and derivatives thereof in particular lactose, such as lactose monohydrate or water-free lactose, dextrose, sorbitol, mannitol, saccharose, maltodextrin, isomalt, or celluloses like microcrystalline cellulose or powdered celluloses and the like. The diluents may present in an amount from about 10% to 80% by weight of composition, preferably from about 40% to 70% by weight of composition.

The disintegrants used in the pharmaceutical composition of the present invention are selected from the group consisting of a sodium starch glycolate, alginates, pregelatinized starch, croscarmellose and cross-linked PVP like collidone and crospovidone or the like. According to the present invention, preferable disintegrants are crospovidone and sodium starch glycolate. The disintegrants may present in an amount from about 1% to 10% by weight of composition, preferably from about 4% to 8% by weight of composition.

According to present invention, glidants present in the pharmaceutical dosage form are such as silicon dioxide, talc, magnesium stearate or the like. A preferred glidant is silicon dioxide and may present in amount from about 0.1% to 10% by weight of composition.

According to present invention, lubricants present in the pharmaceutical dosage form are such as fatty acids or fatty acid derivatives, such as alkali and earth alkali salts of stearic, lauric and/or palmitic acid. A preferred lubricant is magnesium stearate and may present in amount from about 0.1% to 10% by weight of composition.

The pharmaceutical composition of the present invention may be present in a pharmaceutically administrable dosage form including: tablets, capsules, pellets, granules, sachets, or the like.

According to another embodiment of the present invention, there is provided a pharmaceutical composition comprising from about 1% to about 10% w/w of phytonadione, from about 10% to about 30% w/w of dibasic calcium phosphate, from about 10% to about 80% w/w of diluents, from about 1% to about 10% w/w of binders, from about 1% to about 12% w/w of disintegrants, from about 0.1% to about 10% w/w of lubricants, from about 0.1% to about 10% w/w of glidants and optionally from about 1% to about 10% w/w of a film coating substance.

In particular, the present invention provides a pharmaceutical composition comprising about 4% w/w of phytonadione, about 17% w/w of dibasic calcium phosphate, about 57% w/w of lactose monohydrate, about 4% w/w of acacia gum, about 5% to about 10% w/w of starch, about 5% w/w of colloidal silicon dioxide, about 0.8% w/w of talc, and about 0.4% w/w of magnesium stearate and optionally from about 1% to about 10% w/w of the film coating substance.

According to another embodiment of the present invention, the pharmaceutical composition of the present invention can be prepared by a process comprising the steps of—preparing a dispersion of phytonadione with aqueous solvent/organic solvent in presence of binder; preparing a granules/powder material by spraying a dispersion of phytonadione onto the mixture of pharmaceutically acceptable excipients; compressing/filling the prepared granules/powder materials to form a composition and optionally coating the composition.

A second aspect of the present invention provides a process for the preparation of a pharmaceutical composition of the present invention, wherein the process comprises the steps of: preparing a dispersion of phytonadione by dissolving acacia binder into water and adding phytonadione into the dispersion; shifting intra-granular materials of one or more pharmaceutically acceptable excipients through sieve and loading into fluidized bed granulator; spraying a phytonadione dispersion onto intra-granular materials to form granules; further lubricating the granules; and directly compressing the lubricated granules into tablets or filling the lubricated granules into capsule dosage form.

According to another embodiment of the present invention, the pharmaceutical composition of the present invention exhibits improved bioavailability in comparison to marketed pharmaceutical composition of phytonadione, MEPHYTON.

The present invention pharmaceutical composition shows mean Cₘₐₓ of about 10±0.5 ng/ml to 15±0.5 ng/ml, mean AUC₀₋ₜ of about 80±0.5 hr*ng/ml to 85±0.5 hr*ng/ml and mean AUC_{0-inf} of about 82±0.5 hr*ng/ml to 87±0.5 hr*ng/ml for phytonadione.

In particular, the present invention pharmaceutical composition shows mean Cₘₐₓ of at least 11.5±0.5 ng/ml, mean AUC₀₋ₜ of at least 82.1±0.5 hr*ng/ml and mean AUC_{0-inf} of at least 85.8±0.5 hr*ng/ml of phytonadione.

In yet another embodiment of the present invention, the pharmaceutical composition remains stable for longer period of time in different thermo-hygrostats 25° C./60%±5% RH, 30° C./65%±5% RH, and 40° C./75%±5% RH as per ICH guidelines.

In particular, the present invention provides, the pharmaceutical composition remains stable with less than 1.0% of impurity, preferably 0.9% of impurity for at least up to six months at 25° C./60%±5% RH, 30° C./65%±5% RH, and 40° C./75%±5% RH.

The present invention is illustrated below by reference to the following example. However, one skilled in the art will appreciate that the specific methods and results discussed are merely illustrative of the invention, and not to be construed as limiting the invention, as many variations thereof are possible without departing from the spirit and scope of the invention.

### Example 1

TABLE 1

| | | T1 | | T2 | |
|---|---|---|---|---|---|
| Batch No | | Mg/ | % W/ | Mg/ | % W/ |
| No. | Excipients | Tab | W | Tab | W |

Dry Mixing

| | | | | | |
|---|---|---|---|---|---|
| 1. | Lactose Monohydrate | 70.50 | 57.29 | 70.50 | 57.29 |
| | (Granulac 200) | | | | |
| 2. | Dibasic Calcium | 21.00 | 17.21 | 21.00 | 17.21 |
| | Phosphate | | | | |
| 3. | Pregelatinized starch | 5.00 | 4.10 | 5.00 | 4.10 |

Binder-I

| | | | | | |
|---|---|---|---|---|---|
| 4. | Purified water | Q.s | -NA- | Q.s | -NA- |

Binder-II

| | | | | | |
|---|---|---|---|---|---|
| 5. | Phytonadione* | 5.00 | 4.10 | 5.00 | 4.10 |
| 6. | Acacia Gum | 5.00 | 4.10 | 5.00 | 4.10 |
| 7. | Purified water | Q.S | -NA- | Q.S | -NA- |

Extra Granular

| | | | | | |
|---|---|---|---|---|---|
| 8. | Starch 5% Maize | 7.00 | 5.74 | 7.00 | 5.74 |
| 9. | Colloidal Silicon | 7.00 | 5.74 | 7.00 | 5.74 |
| | Dioxide | | | | |
| 10. | Talc | 1.00 | 0.82 | 1.00 | 0.82 |

Lubrication

| | | | | | |
|---|---|---|---|---|---|
| 11. | Magnesium stearate | 0.50 | 0.41 | 0.50 | 0.41 |
| | Total | 122.00 | 100.00 | 122.00 | 100.00 |

*Phytonadione used in batch T1 is having Z isomer content of 12.91% and batch T2 is having Z isomer content of 17.05%.

Procedure:

1. Sift intra-granular material through 30# sieve and load it in FBP (Fluidized Bed Processor).

2. Prepare binder solution by dissolving Acacia in water and disperse phytonadione to it.

3. Granulate the intra-granular material loaded in Fluidized Bed Processor with prepared binder solution and dry the granules to get desired LOD of granules.

4. Sift dried granules through 30# sieve and mix with extra granular material in V cone blender. Lubricate the blended material with 40# sifted magnesium stearate in V cone blender.

5. Compress the lubricated blend using suitable punches into tablet dosage form.

Drug Release Profile Study:

The drug release profile of stable solid pharmaceutical dosage form of phytonadione as described in Example 1 is depicted in Table 2.

TABLE 2

| Batch # | | Test | Test |
|---|---|---|---|
| | Time | Formulation (T1) | Formulation (T2) |
| Sr.# | (min) | % Drug Released | |
| 1. | 10 | 88(2.6) | 90(1.3) |
| 2. | 15 | 92(1.9) | 95(0.7) |
| 3. | 20 | 94(1.4) | 97(1.1) |
| 4. | 30 | 96(1.1) | 99(1.4) |
| 5. | 45 | 98(2.0) | 99(1.4) |
| 6. | 60 | 97(0.5) | 100(2.4) |

| | |
|---|---|
| Condition | pH 6.8 Phospahte Buffer + |
| | 1.0% Triton-x-100, 900 ml, |
| | 75 RPM, USP apparatus II |

As per the results mentioned in Table 2, Example 1 formulations T1 and T2 meet the immediate release profile criteria of disintegration and dissolution profile of the present invention.

Example 1 formulations T1 and T2 show more than 80% drug release in 15 minutes in 900 ml of dissolution media containing mixture of pH 6.8 phosphate Buffer and 1.0% Triton-x-100 at 75 RPM using USP dissolution apparatus II.

Stability Study:

The solid pharmaceutical compositions of example 1 are studied for stress conditions to determine stability of the formulations as per ICH guidelines. The tablets are individually placed in different thermo-hygrostats 25° C./60%±5% RH, 30° C./65%±5% RH, and 40° C./75%±5% RH.

The amount of drug substance and presence of impurities are measured using developed and validated HPLC method at an interval of 1, 3 and 6 months.

Table 3 shows the results of stability study of phytonadione solid pharmaceutical composition as below.

TABLE 3

| | Stability Conditions as per ICH | | | |
|---|---|---|---|---|
| | Time | 40° C./ | 25° C./ | 30° C./ |
| | Duration | 75% RH | 60% RH | 65% RH |
| Drug | 1 Month | 101.6 | 100.4 | 101.3 |
| substances | 3 Month | 97.77 | 99.73 | 99.42 |
| | 6 month | 97.8 | 100.2 | 100.9 |
| Impurities | 1 Month | 0.657 | 0.777 | 0.725 |
| | 3 Month | 0.78 | 0.79 | 0.57 |
| | 6 month | 0.55 | 0.61 | 0.59 |

The above table 3 result shows not more than 0.8% impurities measured after 1 month, 3 months and 6 months intervals at 25° C./60%±5% RH, 30° C./65%±5% RH, and 40° C./75%±5% RH conditions and hence remains stable for a longer period of time.

Oral Bioavailability Study:

An open label, balanced, randomized, single-dose, three-treatment, three-sequence, three-period, crossover oral bioequivalence study of phytonadione tablets USP, 5 mg and MEPHYTON (PHYTONADIONE) Tablets 5 mg are conducted to evaluate pharmacokinetic parameters and single-dose oral bioavailability of phytonadione tablets USP, 5 mg as compared to MEPHYTON (PHYTONADIONE) Tablets 5 mg. The product label for MEPHYTON list the following inactive ingredients: acacia (UNII: 5C5403N26O); calcium phosphate (UNII: 97Z1WI3NDX); silicon dioxide (UNII: ETJ7Z6XBU4); lactose (UNII: J2B2A4N98G); magnesium stearate (UNII: 70097M6I30), starch, Corn (UNII: O8232NY3SJ); and talc (UNII: 7SEV7J4R1U).

Healthy, willing, volunteers aged between 18 and 45 years (both inclusive) are selected on the basis of laboratory evaluations during screening, medical history, clinical examination (including physical examination and systemic examination), Chest X-ray (PA view), ECG recordings.

Table 4 shows result of oral bioavailability study data of T1 formulation as below.

TABLE 4
Test Formulation (T1)

| | Cₘₐₓ | AUC₀₋ₜ | AUC_{0-inf} |
|---|---|---|---|
| Parameters | (ng/mL) | (hr*ng/mL) | (hr*ng/mL) |
| N | 23 | 23 | 23 |
| Mean | 11.502 | 82.118 | 85.855 |
| SD | 5.7433 | 37.8624 | 37.9415 |
| Minimum | 2.90 | 20.98 | 24.14 |
| Median | 11.014 | 86.569 | 96.191 |
| Maximum | 26.43 | 145.97 | 147.92 |
| CV (%) | 49.93 | 46.11 | 44.19 |
| Geometric Mean | 10.102 | 72.071 | 76.253 |

Table 5 shows result of oral bioavailability study data of T2 formulation as below.

TABLE 5
Test Formulation (T2)

| | Cₘₐₓ | AUC₀₋ₜ | AUC_{0-inf} |
|---|---|---|---|
| Parameters | (ng/mL) | (hr*ng/mL) | (hr*ng/mL) |
| N | 23 | 23 | 23 |
| Mean | 12.433 | 95.507 | 98.993 |
| SD | 7.0219 | 56.0658 | 56.1814 |
| Minimum | 0.49 | 2.81 | 4.30 |
| Median | 13.288 | 98.436 | 104.595 |
| Maximum | 25.00 | 206.83 | 209.57 |
| CV (%) | 56.48 | 58.70 | 56.75 |
| Geometric Mean | 9.643 | 72.584 | 77.424 |

Table 6 shows result of oral bioavailability study data of reference formulation as below.

TABLE 6
Reference Formulation (R)

| | Cₘₐₓ | AUC₀₋ₜ | AUC_{0-inf} |
|---|---|---|---|
| Parameters | (ng/mL) | (hr*ng/mL) | (hr*ng/mL) |
| N | 23 | 23 | 23 |
| Mean | 8.001 | 51.826 | 56.140 |
| SD | 8.0422 | 46.1529 | 45.5550 |
| Minimum | 0.89 | 2.38 | 2.79 |
| Median | 5.547 | 34.929 | 38.433 |
| Maximum | 38.20 | 196.80 | 200.10 |
| CV (%) | 100.52 | 89.05 | 81.15 |
| Geometric Mean | 5.583 | 35.173 | 40.294 |

Table 7 shows statistical data of oral bioavailability study of reference, and test formulations (T1 & T2).

TABLE 7

| ANOVA p-value | Reference | T1 | T2 |
|---|---|---|---|
| Ln-transformed | 0.0012 | 0.0003 | 0.0005 |
| Formulation | | | |
| Sequence | 0.5409 | 0.3338 | 0.3724 |
| Period | 0.2888 | 0.8807 | 0.9926 |

The above results as shown in table 4, 5, 6 & 7 depicts about comparative oral bioavailability study data of both test formulations T1 and T2 and the reference formulation. The result shows an increased in mean Cₘₐₓ (ng/mL), mean AUC₀₋ₜ(hr*ng/mL) and mean AUC_{0-inf} (hr*ng/mL) of both the test formulations T1 and T2 in comparison to the reference formulation which ultimately indicates an increase in bioavailability of current claim invention in comparison to marketed phytonadione formulation.

What is claimed:

1. A process of preparing a stable pharmaceutical composition of phytonadione comprising: (a) preparing a dispersion of phytonadione in a pharmaceutically acceptable binder; (b) mixing or spraying the dispersion with one or more pharmaceutically acceptable excipients to form the stable pharmaceutical composition; and (c) formulating the stable pharmaceutical composition obtained from step (b) into a pharmaceutically administrable dosage form.

2. The process of preparing a stable pharmaceutical composition according to claim 1, wherein the pharmaceutically acceptable binder is selected from the group consisting of: natural binders, synthetic binders, semisynthetic binders, and a combination thereof.

3. The process of preparing a stable pharmaceutical composition according to claim 2, wherein the pharmaceutically acceptable binder is polyvinylpyrrolidone, starch, hydroxypropylmethyl cellulose, hydroxyethyl cellulose, hydroxypropyl cellulose, polyethylene glycol, acacia, gelatine, tragacanth, or alginic acid.

4. The process of preparing a stable pharmaceutical composition according to claim 1, wherein a ratio by weight of phytonadione to acacia binder in the stable pharmaceutical composition is in the range of from about 1:2 to about 1:0.1.

5. The process of preparing a stable pharmaceutical composition according to claim 1, wherein the pharmaceutically administrable dosage form is tablets, capsules, pellets, granules, or sachets.

6. The process of preparing a stable pharmaceutical composition according to claim 1, wherein the stable pharmaceutical composition exhibits improved bioavailability in comparison to a marketed pharmaceutical composition.

7. The process of preparing a stable pharmaceutical composition according to claim 6, wherein the stable pharmaceutical composition exhibits mean Cₘₐₓ of at least 11.5±0.5 ng/ml.

8. The process of preparing a stable pharmaceutical composition according to claim 6, wherein the stable pharmaceutical composition exhibits mean AUC₀₋ₜ of at least 82.1±0.5 hr*ng/ml.

9. The process of preparing a stable pharmaceutical composition according to claim 6, wherein then said pharmaceutical composition exhibits mean AUC_{0-inf} of at least 85.8±0.5 hr*ng/ml.

10. The process of preparing a stable pharmaceutical composition according to claim 1, wherein the stable pharmaceutical composition exhibits improved bioavailability in comparison to a comparative pharmaceutical composition that was made without using a dispersion of phytonadione in a pharmaceutically acceptable binder.

11. The process of preparing a stable pharmaceutical composition according to claim 10, wherein the stable pharmaceutical composition exhibits mean Cₘₐₓ of at least 11.5±0.5 ng/ml.

12. The process of preparing a stable pharmaceutical composition according to claim 10, wherein the stable pharmaceutical composition exhibits mean AUC₀₋ₜ of at least 82.1±0.5 hr*ng/ml.

13. The process of preparing a stable pharmaceutical composition according to claim 10, wherein then said pharmaceutical composition exhibits mean AUC_{0-inf} of at least 85.8±0.5 hr*ng/ml.

14. A pharmaceutical composition comprising from about 1% to about 10% w/w of phytonadione, from about 10% to about 30% w/w of dibasic calcium phosphate, from about 10% to about 80% w/w of diluents, from about 1% to about 10% w/w of binders, from about 1% to about 12% w/w of disintegrants, from about 0.1% to about 10% w/w of lubricants, from about 0.1% to about 10% w/w of glidants, and optionally from about 1% to about 10% w/w of a film coating substance.

15. The pharmaceutical composition according to claim 14 comprising about 4% w/w of phytonadione, about 17% w/w of dibasic calcium phosphate, about 57% w/w of lactose monohydrate, about 4% of acaia gum, about 5% w/w of starch, about 5% w/w of colloidal silicon, about 0.8% w/w of talc, about 0.4% w/w of magnesium stearate, and from about 1% to about 10% w/w of the film coating substance.
